(19) United States
(12) Patent Application Publication
(10) Pub. No.: US 2008/0146671 A1
(43) Pub. Date: Jun. 19, 2008

(54) MICROEMULSION PRECONCENTRATE COMPRISING A RENIN INHIBITOR

(76) Inventor: Isabel Ottinger, Freiburg (DE)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/034,406

(22) Filed: Feb. 20, 2008

Related U.S. Application Data
(63) Continuation of application No. 10/580,258, filed on May 24, 2006, now abandoned.
(60) Provisional application No. 60/531,562, filed on Dec. 19, 2003.
(60) Provisional application No. 60/547,676, filed on Feb. 25, 2004.

(51) Int. Cl. A61K 31/16; A61P 9/00

(52) U.S. Cl. 514/616

(57) ABSTRACT

The present invention relates to pharmaceutical compositions for oral administration comprising a δ-amino-γ-hydroxy-ω-aryl-alkanoic acid amide renin inhibitors as the active ingredient. In particular, the present invention relates to galenic formulations in the form of microemulsion preconcentrates comprising the active ingredient and at least one absorption enhancing excipient which preconcentrates provide spontaneously dispersible water-in-oil microemulsions which upon further dilution in aqueous medium, e.g., gastric fluids, convert to oil-in-water microemulsions. The present invention also relates to the processes for their preparation and to their use as medicaments.

This application is a Continuation Application of application Ser. No. 10/580,258, filed May 24, 2006, which is a 371 of PCT/EP2004/-14433, filed Dec. 17, 2004, which claims benefit of Provisional Application No. 60/547,676, filed Feb. 25, 2004 and Provisional Application No. 60/531,562, filed Dec. 19, 2003.

The present invention relates to pharmaceutical compositions for oral administration comprising a δ-amino-γ-hydroxy-ω-aryl-alkanoic acid amide derivative as the active ingredient, e.g., those disclosed in U.S. Pat. No. 5,559,111, the entire contents of which are incorporated herein by reference. In particular, the present invention relates to galenic formulations in the form of a microemulsion preconcentrate comprising the active ingredient and at least one absorption enhancing excipient which preconcentrates provide spontaneously dispersible water-in-oil (w/o) microemulsions which upon further dilution in aqueous medium, e.g., gastric fluids, convert to oil-in-water (o/w) microemulsions. The present invention also relates to the processes for their preparation and to their use as medicaments.

The δ-amino-γ-hydroxy-ω-aryl-alkanoic acid amide derivatives are a class of potent renin inhibitors which present highly specific difficulties in relation to administration generally and galenic formulation in particular, including problems of drug bioavailability and variability in inter- and intra-subject dose response thus necessitating development of a non-conventional dosage form.

There are many advantages to the use of a microemulsion over a conventional emulsion (or macroemulsion) for oral drug delivery. Microemulsions form spontaneously, without the need for a high input of energy and are therefore easy to prepare and scale up for commercial applications; they have thermodynamic stability due to their small particle size and therefore have a long shelf life; they have an isotropically clear appearance so that they may be monitored by spectroscopic means; they have a relatively low viscosity and are therefore easy to transport and mix; they have a large interfacial area which accelerates surface reactions; they have a low interfacial tension which permits its flexible and high penetrating power and, lastly, they offer the possibility of improved drug solubilization and protection against enzymatic hydrolysis. In addition, microemulsions may undergo phase inversion upon addition of an excess of the dispersed phase or in response to a temperature change and this is a property of these systems that can affect drug release from microemulsions both in vitro and in vivo. For instance, as described in U.S. Pat. No. 5,633,226, a w/o microemulsion containing, e.g., a water-soluble drug in the internal hydrophilic phase, upon administration directly to the body of an animal, including human, the body fluids themselves are sufficient to convert the w/o microemulsion to an o/w microemulsion, thereby slowly releasing the drug in situ. This is particularly advantageous over pre-conversion with water in that because body fluids are employed, the total volume of liquid administered is smaller. This method is particularly useful in administration of such drugs as peptides, proteins, or other molecules with bonds that are readily attacked by enzymes, where the oil protects the drug until it is slowly released as the body fluids convert the emulsion.

The use of lipid-based microemulsions to enhance the bioavailability of different drugs, including peptides, has already been described, e.g., in GB 2,222,770 and International PCT Patent Application No. WO 94/08605. Thus, GB 2,222,770 discloses microemulsions and corresponding microemulsion preconcentrates for use with the highly hydrophobic cyclosporin peptides. Accordingly, a suitable preconcentrate comprises 1,2-propylene glycol as the hydrophilic component, a caprylic-capric acid triglyceride as the lipophilic component and a mixture of a polyoxyethylene glycolated hydrogenated castor oil and glycerin monooleate (ratio 11:1) as the surfactant-cosurfactant. Such formulations may then be diluted with water, to give o/w rather than w/o microemulsions. WO 94/08605 describes self-emulsifying w/o microemulsions which comprise (i) a lipophilic phase in which the oil and the low HLB surfactant are a physical mixture of medium and long chain fatty acid components; (ii) a high HLB surfactant; and (iii) an aqueous hydrophilic phase comprising a water soluble therapeutic agent.

Microemulsions are typically a slightly opaque, opalescent, non-opaque or substantially non-opaque colloidal dispersion that are formed spontaneously or substantially spontaneously when the components are brought into contact with an aqueous medium. A microemulsion is thermodynamically stable and typically contains dispersed droplets of a mean diameter less than about 200 nm (2000 Å). Generally, microemulsions comprise droplets or liquid nanoparticles that have a mean diameter of less than about 150 nm (1500 Å), typically less than 100 nm, generally greater than 10 nm, and they are stable over periods up to 24 hours.

The formation of microemulsions usually involves a combination of three or more components, e.g., a hydrophilic phase such as water or polyethylene glycol, a lipophilic phase such as an oil and surfactant(s). The tendency to form either a w/o or an o/w microemulsion is influenced by the properties of the lipophilic phase and the surfactant(s).

A microemulsion preconcentrate is defined herein as being a composition which spontaneously forms a microemulsion in an aqueous medium, e.g., in water, e.g., upon dilution ranging from about 1:1 to about 1:300, preferably from about 1:1 to about 1:70, more preferably from about 1:1 to about 1:10, or in the gastric juices after oral administration. Preferably, the microemulsion preconcentrates of the present invention comprise a hydrophilic phase, a lipophilic phase and a surfactant which upon admixing form, e.g., a stable w/o microemulsion or other micellar composition.

Surfactants are conveniently classified on an empirical scale known as the hydrophilic-lipophilic balance (HLB) which runs from 1 to 20. In general, w/o microemulsions are formed using surfactants (or emulsifiers) which have an HLB value in the range of about 2.5 to 6 whilst o/w microemulsions are formed using surfactants which have an HLB value ranging from about 8 to about 18. It has long been recognized that low interfacial tension contributes to the thermodynamic stability of microemulsions. General reviews of microemulsions may be found, e.g., as described by Kahlweit in Science, 240, 617-621 (1988).

The role of a cosurfactant, usually a short-chain alcohol, is to increase the interfacial fluidity by penetrating the surfactant film and consequently creating a disordered film due to the void space among surfactant molecules. The use of a cosurfactant in microemulsions is however optional and alcohol-free self-emulsifying emulsions and microemulsions have been described in the literature, e.g., by Pouton et al. in Int. Journal of Pharmaceutics, 27, 335-348 (1985) and by Osborne et al. in J. Disp. Sci. Tech., 9, 415-423 (1988).

In accordance with the present invention it has now been found that stable pharmaceutical compositions with δ-amino-γ-hydroxy-ω-aryl-alkanoic acid amide renin inhibitors, having particularly interesting bioavailability characteristics and reduced variability in inter- and intra-subject bioavailability parameters, are obtainable as microemulsion preconcentrates, in particular, as w/o preconcentrates. The compositions of the present invention comprise at least one excipient that enhance the oral absorption of the active ingredient either by inhibition of efflux or by enhancing transcellular absorption, e.g., by increasing membrane fluidity, and thereby would substantially reduce the difficulties encountered previously. It has been shown that the compositions in accordance with the present invention may enable effective dosaging with concomitant enhancement as well as reduced variability of absorption/bioavailability levels for and between individual subjects. Thus, the invention may achieve effective therapy with tolerable dosage levels of such δ-amino-γ-hydroxy-ω-aryl-alkanoic acid amide derivatives, and may permit closer standardization and optimization of daily dosage requirements for each individual. Consequently, occurrence of potential undesirable side-effects may be diminished and overall cost of therapy may be reduced.

The δ-amino-γ-hydroxy-ω-aryl-alkanoic acid amide derivatives to which the present invention applies are any of those having renin inhibitory activity and, therefore, pharmaceutical utility, e.g., as therapeutic agents for the treatment of hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth, hyperaldosteronism, cognitive impairment, alzheimers, dementia, anxiety states and cognitive disorders.

Accordingly, the present invention provides a pharmaceutical composition comprising a δ-amino-γ-hydroxy-ω-aryl-alkanoic acid amide renin inhibitor as the active ingredient in an absorption enhancing carrier medium comprising:

(a) a lipophilic component;
(b) a high HLB surfactant; and
(c) a hydrophilic component;
which composition upon admixing forms a stable microemulsion preconcentrate.

Preferably, the lipophilic component comprises a low HLB surfactant.

More preferably, the lipophilic component comprises a low HLB surfactant which is based on a medium or a long chain fatty acid, or a mixture of fatty acids thereof, and an oil which is a medium or a long chain fatty acid triglyceride, or a mixture of triglycerides thereof.

Most preferably, the lipophilic component comprises a low HLB surfactant which is based on a medium chain fatty acid, or a mixture of fatty acids thereof, and an oil which is a medium chain fatty acid triglyceride, or a mixture of triglycerides thereof.

Preferably, the medium chain fatty acids of the lipophilic component have from 8 to 12 carbon atoms.

Advantageously, the components of the absorption enhancing carrier medium of the present invention may all be composed of absorption enhancing excipients. However, only one absorption enhancing component may be sufficient, e.g., the high HLB surfactant.

Preferably, the active ingredient is dissolved in the hydrophilic component of the carrier medium to form a pharmaceutical composition which upon admixing forms a stable microemulsion preconcentrate. Preferably, the microemulsion preconcentrate of the present invention is in the form of a w/o microemulsion which upon administration or dilution with an aqueous medium spontaneously converts to an o/w microemulsion.

Preferably, a δ-amino-γ-hydroxy-ω-aryl-alkanoic acid amide renin inhibitor of the present invention has the formula

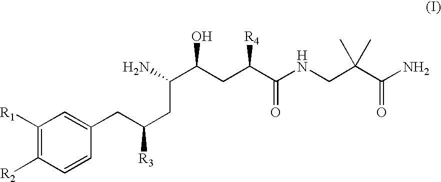

wherein R₁ is C₁₋₄alkoxy-C₁₋₄alkoxy or C₁₋₄alkoxy-C₁₋₄alkyl; R₂ is C₁₋₄alkyl or C₁₋₄alkoxy; and R₃ and R₄ are independently branched C₃₋₄alkyl; or a pharmaceutically acceptable salt thereof.

More preferably, the δ-amino-γ-hydroxy-ω-aryl-alkanoic acid amide renin inhibitor of the present invention is a compound of formula (I) wherein R₁ is 3-methoxypropoxy; R₂ is methoxy; and R₃ and R₄ are isopropyl; or a pharmaceutically acceptable salt thereof.

Most preferably, the δ-amino-γ-hydroxy-ω-aryl-alkanoic acid amide renin inhibitor of the present invention is (2S,4S,5S,7S)-5-amino-4-hydroxy-2-isopropyl-7-[4-methoxy-3-(3-methoxy-propoxy)-benzyl]-8-methyl-nonanoic acid (2-carbamoyl-2-methyl-propyl)-amide hemifumarate, also known as aliskiren.

In accordance with the present invention the active ingredient may be present in an amount by weight of up to about 25% by weight of the total composition of the present invention, e.g., from about 0.1% by weight. The active ingredient is preferably present in an amount of 0.5 to 15% by weight of the composition.

Listed below are definitions of various terms used herein to describe the carrier medium of the pharmaceutical compositions of the present invention. The preferred embodiments are to be construed as merely illustrative and not a limitation of the scope of the present invention in any way, and further information and examples may be found, e.g., in Rowe et al., “Handbook of Pharmaceutical Excipients”, 4th Edition, Pharmaceutical Press, London, Chicago (2003).

The term “medium chain fatty acid” as used herein refers to a fatty acid moiety having from 6 to 12, preferably from 8 to 12 carbon atoms, which may be branched or unbranched, preferably unbranched, and which may be optionally substituted.

The term “long chain fatty acid” as used herein refers to a fatty acid moiety which may be saturated, mono-unsaturated or poly-unsaturated, having from 14 to 22, preferably from 14 to 18, carbon atoms which may be branched or unbranched, preferably unbranched, and which may be optionally substituted.

Suitable medium and long chain fatty acid triglycerides for use in the present invention may be of natural, semi-synthetic or synthetic origin and may include blends of different fatty acid triglycerides. Suitable triglycerides for use herein are readily available from commercial suppliers.

Preferred medium chain fatty acid triglycerides for use herein are, e.g., saturated medium chain fatty acid triglycerides available under the trade names ACOMED, MYRITOL, CAPTEX, NEOBEE M 5 F, MIGLYOL 810, MIGLYOL 812, MIGLYOL 818, MAZOL, SEFSOL 860 and SEFSOL 870.

Especially useful medium chain fatty acid triglycerides include caprylic (C₈) acid optionally admixed with capric (C₁₀) acid, for instance from 50 to 100% (w/w) of caprylic acid and from 0 to 50% (w/w) of capric acid triglycerides. Suitable examples include, e.g., CAPTEX 355, CAPTEX 200, CAPTEX 350, CAPTEX 850, CAPTEX 800, CAPTEX 8000, MIGLYOL 810, MIGLYOL 812 and MIGLYOL 818 (which also comprises a linoleic acid triglyceride). Preferred medium chain fatty acid triglycerides are CAPTEX 200 and MIGLYOL 812.

Suitable long chain fatty acid triglycerides may be conveniently obtained from neutral plant, vegetable and fish oils such as shark oil, olive oil, sesame oil, peanut oil, castor oil, safflower oil, sunflower oil and soybean oil which may be in their natural state or partially or fully hydrogenated. Soybean oil consists of oleic acid (25%), linoleic acid (54%), linolenic acid (6%), palmitic acid (11%) and stearic acid (4%) triglycerides whilst safflower oil consists of oleic acid (13%), linoleic acid (76%), stearic acid (4%) and palmitic acid (5%) triglycerides. Suitably in such long-chain fatty acid triglycerides, the major fatty acid components are C₁₈-saturated, monounsaturated or polyunsaturated fatty acids, preferably C₁₈-monounsaturated or polyunsaturated fatty acids.

It will be appreciated that when so required, mixtures of medium and long chain fatty acid triglycerides are obtained by physically admixing triglycerides which essentially have medium chain fatty acid moieties with triglycerides which essentially have long chain fatty acid moieties, to create artificial mixtures of medium and long chain fatty acid triglycerides in the desired ratios.

Suitable low HLB surfactants for use in the present invention include, put are not limited to, fatty acid mono- and diglycerides, as well as mixtures thereof, and may also comprise a small amount by weight of free fatty acid. The mono- and diglycerides may each include blends of different fatty acid mono- and diglycerides.

Suitable medium chain fatty acid mono- and diglycerides are formed from caprylic and capric acids. Suitable blends comprise from about 50 to 100% caprylic acid and from about 0 to about 50% capric acid mono and/or diglycerides. Suitable commercial sources of these include, but are not limited to, absorption enhancing low HLB surfactants available under the trade name CAPMUL (Karlsham Lipid Specialties, Columbus Ohio), e.g, CAPMUL MCM which comprises monoglycerides (77.4%), diglycerides (21%) and free glycerol (1.6%), with a fatty acid composition of caproic acid (3.2%), caprylic acid (66.8%), capric acid (29.6%), lauric acid (0.3%) and palmitic acid (0.1%) and CAPMUL MCM C8 which has monoglycerides (70-90%), diglycerides (10-30%) and free glycerol (2-4%), with a fatty acid composition which comprises at least 98% of caprylic acid (manufacturers data).

Suitable long chain fatty acid monoglycerides include glycerol monooleate, glycerol monopalmitate and glycerol monostearate. Suitable commercially available examples of such include the products available under the trade names MYVEROL, such as MYVEROL 18-92 and 18-99, MYVATEX and MYVAPLEX. Another useful long chain fatty acid monoglyceride containing product is ARLACEL 186 which includes, in addition to glycerol monooleate, propylene glycol (10%). The main fatty acids of MYVEROL 18-99 are oleic acid (61%), linoleic acid (21%), linolenic acid (9%) and palmitic acid (4%). Suitably in such long chain monoglycerides, the major fatty acid component is a C₁₈-saturated, monounsaturated or polyunsaturated fatty acid, preferably a C₁₈-monounsaturated or polyunsaturated fatty acid. In addition, diacetylated and disuccinylated versions of the monoglycerides such as the product available under the trade name MYVATEX SMG may also be useful.

Propylene glycol monofatty acid esters may also be used. The fatty acid constituent may include both saturated and unsaturated fatty acids having, preferably, 8 to 12 carbon atoms. Particularly suitable are propylene glycol mono esters of caprylic and lauric acid as commercially available, e.g., under the trade names SEFSOL 218, CAPRYOL 90 and LAUROGLYCOL 90, from, e.g., Nikko Chemicals Co., Ltd. or Gattefosse, or CAPMUL PG-8 from Abitec.

Preferably, the low HLB surfactant will have an HLB value in the range of from about 2.5 to about 6, e.g., the HLB value of CAPMUL MCM is about 5.5.

Suitably, the lipophilic phase comprising the oil and the low HLB surfactant together may be present from about 15 to about 80% by weight of the total composition of the present invention, preferably, from about 20 to about 70% by weight and, more preferably, from about 30% to about 60% by weight.

The high HLB surfactants suitable for use in the present invention include, but are not limited to, non-ionic efflux inhibiting and thereby absorption enhancing surfactants such as:
(a) Polyoxyethylene fatty acid esters, e.g., polyoxyethylene stearic acid esters of the type available under the trade name MYRJ, e.g., MYRJ 52 (a polyoxyethylene 40 stearate). Other related products include polyethoxylated saturated hydroxy fatty acids which may be produced by reacting a saturated hydroxy fatty acid, e.g., C₁₈ to C₂₀ fatty acid, with ethylene oxide or polyethylene glycol. Suitable examples for the present invention include those known in the art and commercially available, e.g., from the BASF company under the trade mark SOLUTOL. Especially preferred is SOLUTOL HS15 which is known, e.g., from the BASF technical leaflet MEF 151E (1986), to comprise of about 70% polyethoxylated 12-hydroxystearate by weight and about 30% by weight unesterified polyethylene glycol component;
(b) Polyoxyetheylene-sorbitan fatty acid esters (polysorbates), e.g., the mono- and trilauryl, palmityl, stearyl and oleyl esters, for instance the polyoxyethylene sorbitan monooleates available under the trade name of TWEEN, such as TWEEN 20, 21, 40, 60, 61, 65, 80, 81 and 85, of which class TWEEN 80 (polysorbat 80) is especially preferred;
(c) Reaction products of a natural or hydrogenated castor oil and ethylene oxide. The natural or hydrogenated castor oil may be reacted with ethylene oxide in a molar ratio of from about 1:35 to about 1:60, with optional removal of the polyethyleneglycol component from the products. Vadous such surfactants are commercially available. Particularly suitable surfactants include polyethyleneglycol-hydrogenated castor oils available under the trade name CREMOPHOR, e.g., CREMOPHOR RH 40 (polyoxyl 40 hydrogenated castor oil) and CREMOPHOR EL (polyoxyl 35 castor oil);
(d) Polyoxyethylene-polyoxypropylene co-polymers and block co-polymers, poloxamers, e.g., of the type known and commercially available under the trade names PLURONIC, LUTROL and MONOLAN. An especially preferred product of this class is PLURONIC F68 (poloxamer 188) from BASF, having a melting point of about 52° C. and a molecular weight of about 6800 to 8975;
(e) Polyoxyethylene glycol long-chain alkyl ethers, such as polyoxyethylated glycol lauryl ether;
(f) Polyoxyethylene glycol long-chain alkyl esters, such as PEG-monostearate; and
(g) Water soluble tocopheryl polyethylene glycol succinic acid esters (TPGS), e.g., those with a polymerization number ca 1000, e.g., VITAMIN E-TPGS, available from Eastman Fine Chemicals Kingsport, Tex., USA.

For use herein, the high HLB surfactant preferably has an HLB value in the range of 13 to 20.

The high HLB surfactant may comprise from about 5 to about 60% by weight of the total composition of the present invention, preferably, from about 10 to about 50% by weight.

Preferably, the blend of low and high HLB surfactants will have an HLB value in the range of from about 7 to about 15, more preferably from about 8 to about 13.

The hydrophilic component typically has a solubility in water of at least 1 g/100 mL or more, e.g., at least 5 g/100 mL at 25° C. It preferably provides for fast mixing of the active ingredient with water. Such mixing may be determined by routine experimentation, e.g., by various chromatographic methods, e.g., by gas chromatography (GC). Conveniently, the hydrophilic component or phase may also be miscible with an organic solvent, e.g., ether. Generally, the hydrophilic component may comprise an absorption enhancing alcohol, e.g., a water miscible alcohol such as absolute ethanol, glycerol, a glycol such as 1,2-propylene glycol or a polyol such as polyalkylene glycol, a polyalkylene glycol monoether such as transcutol, or a mixture of components thereof. Preferably, the hydrophilic component of the present invention comprises polyalkylene glycol, more preferably, poly(C₂-C₃)-alkyleneglycol. A typical example is polyethylene glycol, e.g., of a preferred molecular weight of 200-1000 daltons, more preferably, 200-400 daltons. Especially preferred hydrophilic component is polyethylene glycol 300 (PEG 300).

The hydrophilic component may be present from about 1 to about 20% by weight of the total composition of the invention, preferably, from about 3 to about 10% by weight.

Preferably the relative proportions of the lipophilic component, the hydrophilic component, and the high HLB surfactant lie within the “Microemulsion” region on a standard three way plot graph. These phase diagrams may be generated in a conventional manner as described, e.g., in GB 2,222,770 and International PCT Patent Application No. WO 96/13273.

The various phases may optionally contain further ingredients, such as, but not limited to:
(a) Lipids, such as phospholipids, in particular lecithins, such as soya bean lecithins, egg lecithin or egg phosphatide, cholesterol or long-chain fatty acids such as oleic acid;
(b) Antioxidants such as n-propyl gallate, butylated hydroxyanisole (BHA) and mixed isomers thereof, δ-α-tocopherol and mixed isomers thereof, ascorbic acid, propylparaben, methylparaben and citric acid (monohydrate), for instance in amounts less than 3, preferably less than 1% (w/w);
(c) Bile salts, for instance as their alkali metal salts, such as sodium taurocholate;
(d) Stabilizers, such as hydroxypropyl cellulose. for instance in amounts less than 3, preferably less than 1% (w/w);
(e) Antimicrobials, such as benzoic acid (sodium salt);
(f) Dioctylsuccinate, di-octylsodium sulfosuccinate or sodium lauryl sulfate;
(g) Propylene glycol mono- and di-fatty acid esters. such as propylene glycol dicaprylate, dilaurate, hydroxystearate, isostearate, laurate, ricinolate, etc. of which the propylene glycol caprylic/capric acid diesters commercially known as Miglyol 840 and Imwitor 408 are especially preferred; and
(h) Protease inhibitors such as aprotinin.

Preferably, the diameter of droplets or particles of o/w microemulsions produced upon dilution of a microemulsion preconcentrate of the present invention, measured, for instance, as the number-average diameter by laser light scattering techniques, is less than 150 nm, more preferably less than 100 nm, yet more preferably less than 50 nm and, most preferably, ranging from about 10 to about 35 nm.

Simple tests, such as dye solubilization, dispersibility in water and conductivity measurements may be used to determine whether the microemulsion is an o/w or a w/o type. A water-soluble dye will disperse in an o/w microemulsion whilst it will remain in its original form in a w/o microemulsion. Likewise, o/w microemulsions are generally dispersible in water whereas w/o microemulsions are generally not. In addition, o/w microemulsions conduct electricity whereas w/o do not. The isotropic nature of the system may be confirmed by examination thereof under polarized light. The microemulsions being micellar in nature are isotropic and therefore non-birefringent when examined under polarized light.

The microemulsion preconcentrates of the present invention, preferably, in the form of a w/o microemulsion, are formed spontaneously or substantially spontaneously when their components are brought into contact, that is without the application of substantial energy supply. For instance, in the absence of high shear energy such as imparted by homogenization and/or microfluidization or other mechanical agitation. Accordingly, the microemulsion preconcentrates may be readily prepared by the simple process of admixing appropriate quantities with gentle mixing or stirring if necessary to ensure thorough mixing. Preferably, the therapeutic agent is dissolved in the hydrophilic phase, either directly or by dilution of a stock solution thereof, and this may then be added to a pre-mixed combination of the oil and the low HLB surfactant with mixing, followed by the high HLB surfactant or vice versa. Alternatively, a drug-free microemulsion preconcentrate may be initially prepared by admixing the oil, the low HLB surfactant, the high HLB surfactant and the hydrophilic component, to which composition may then be added the therapeutic agent. Whilst higher temperatures (40-60° C.) may be needed to solubilize all components during the preparation of the microemulsion preconcentrate, the preferred systems may be formulated at room temperature.

As defined herein, the active ingredient or the therapeutic agent refers to any δ-amino-γ-hydroxy-ω-aryl-alkanoic acid amide derivative which exhibits renin inhibitory activity as may be determined by standard in vitro and in vivo tests known in the art, e.g., by those disclosed in U.S. Pat. No. 5,559,111. The δ-amino-γ-hydroxy-ω-aryl-alkanoic acid amide derivatives may be prepared according to literature procedures, e.g., those described in U.S. Pat. No. 5,559,111.

In a preferred aspect, the present invention provides pharmaceutical compositions in the form of microemulsion preconcentrates comprising at least one absorption enhancing excipient which compositions provide spontaneously dispersible w/o microemulsions which upon further dilution in aqueous medium, e.g., gastric fluids, convert to o/w microemulsions, and a δ-amino-γ-hydroxy-ω-aryl-alkanoic acid amide renin inhibitor which may be orally administered and which will retain its biological activity, thereby overcoming the disadvantages of earlier formulations in which the bioavailability of the δ-amino-γ-hydroxy-ω-aryl-alkanoic acid amide derivatives has been less than satisfactory.

Accordingly, the present invention provides methods for the treatment of hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth, hyperaldosteronism, cognitive impairment, alzheimers, dementia, anxiety states and cognitive disorders, which methods comprise administering a therapeutically effective amount of a pharmaceutical composition as hereinbefore defined to a patient in need thereof.

When required, the pharmaceutical compositions of the present invention are preferably compounded in unit dosage form, e.g., by filling them into orally administrable capsule shells. The capsule shells may be soft or hard gelatin capsule shells. Where the composition is in unit dosage form, each unit dosage will suitably contain from 0.1 to 300 mg of the active ingredient, preferably between 10 and 150 mg of the active ingredient, more preferably between 10 and 100 mg, e.g., 15 mg or 75 mg. Such unit dosage forms are suitable for administration 1 to 5 times daily depending upon the particular purpose of therapy, the phase of therapy and the like. However, if desired, the compositions may be in the form of drink solution and may include water or any other aqueous system, e.g., milk, fruit juice, excluding grapefruit juice, and the like, to provide, e.g., colloidal systems, suitable for drinking, e.g., with a dilution of from about 1:10 to about 1:100.

The present invention further relates to pharmaceutical compositions as described herein above for use as a medicament.

Ultimately, the present invention provides for the use of a fatty acid triglyceride, a low HLB surfactant, a high HLB surfactant, a hydrophilic component and a therapeutic agent as hereinbefore defined in the manufacture of a medicament.

Thus, the present invention relates to use of pharmaceutical compositions as described herein above for the manufacture of a medicament for the treatment of conditions mediated by renin activity, preferably, hypertension, atherosclerosis, unstable coronary syndrome, congestive heart failure, cardiac hypertrophy, cardiac fibrosis, cardiomyopathy postinfarction, unstable coronary syndrome, diastolic dysfunction, chronic kidney disease, hepatic fibrosis, complications resulting from diabetes, such as nephropathy, vasculopathy and neuropathy, diseases of the coronary vessels, restenosis following angioplasty, raised intra-ocular pressure, glaucoma, abnormal vascular growth, hyperaldosteronism, cognitive impairment, alzheimers, dementia, anxiety states and cognitive disorders.

The compositions of the present invention, e.g., those in the illustrative Examples, may show good stability characteristics as indicated by standard stability tests, e.g., having a shelf life stability of up to one, two or three years, and even longer. The compositions of the present invention in form of micellar preconcentrates, in particular w/o preconcentrates, produce stable aqueous micelles, e.g., o/w microemulsions, stable for up to one day or longer.

The above description fully discloses the invention including preferred embodiments thereof. Modifications and improvements of the embodiments specifically disclosed herein are within the scope of the following claims. Without further elaboration, it is believed that one skilled in the art can, using the preceding description, utilize the present invention to its fullest extent. Therefore, the Examples herein are to be construed as merely illustrative and not a limitation of the scope of the present invention in any way.

The microemulsion preconcentrates of the illustrative Examples may generally be prepared by first dissolving the appropriate amount of the therapeutic agent, e.g., aliskiren, in the hydrophilic component, e.g., PEG 300, with stirring if necessary to obtain complete dissolution. The hydrophilic phase containing the drug is then added to the appropriate amounts (by weight) of a mixture of the oil and the low HLB surfactant, to which is then added the high HLB surfactant, with gentle stirring. Alternatively, the hydrophilic phase containing the drug is added to the high HLB surfactant and following upon complete mixing, this is added to the oil plus low HLB surfactant mixture. If necessary, the drug-containing microemulsion preconcentrate is then diluted with the corresponding drug-free microemulsion to adjust the concentration of the drug.

### EXAMPLE 1

| | |
|---|---|
| Aliskiren | 75.00 mg |
| Polysorbat 80 (TWEEN 80) | 212.50 mg |
| PEG 300 | 42.50 mg |
| Propylene glycol di-caprylate/caprate (CAPTEX 200) | 166.67 mg |
| Caprylic acid | 56.67 mg |

### EXAMPLE 2

| | |
|---|---|
| Aliskiren | 75.00 mg |
| Macrogol-15 hydroxystearate (SOLUTOL HS 15) | 233.75 mg |
| PEG 300 | 21.25 mg |
| Glyceryl tri-caprylate/caprate (MIGLYOL 812) | 166.67 mg |
| Caprylic acid | 56.67 mg |

### EXAMPLE 3

| | |
|---|---|
| Aliskiren | 75.00 mg |
| Macrogol-15 hydroxystearate (SOLUTOL HS 15) | 212.50 mg |
| PEG 300 | 21.25 mg |
| Propylene glycol di-caprylate/caprate (CAPTEX 200) | 127.50 mg |
| Caprylic acid | 63.75 mg |

### EXAMPLE 4

| | |
|---|---|
| Aliskiren | 75.00 mg |
| Macrogol-15 hydroxystearate (SOLUTOL HS 15) | 212.50 mg |
| PEG 300 | 21.25 mg |
| Propylene glycol di-caprylate/caprate (CAPTEX 200) | 127.50 mg |
| Mono-/Diglycerides of caprylic acid (CAPMUL MCM C8) | 63.75 mg |

### EXAMPLE 5

| | |
|---|---|
| Aliskiren | 15.00 mg |
| Macrogol-15 hydroxystearate (SOLUTOL HS 15) | 344.75 mg |
| PEG 300 | 49.25 mg |
| Propylene glycol di-caprylate/caprate (CAPTEX 200) | 394.00 mg |
| Caprylic acid | 197.00 mg |

### EXAMPLE 6

| | |
|---|---|
| Aliskiren | 15.00 mg |
| Macrogol-15 hydroxystearate (SOLUTOL HS 15) | 443.25 mg |
| PEG 300 | 49.25 mg |
| Glyceryl tri-caprylate/caprate (MIGLYOL 812) | 328.33 mg |
| Caprylic acid | 164.17 mg |

### EXAMPLE 7

| | |
|---|---|
| Aliskiren | 15.00 mg |
| Macrogol-15 hydroxystearate (SOLUTOL HS 15) | 492.50 mg |
| PEG 300 | 49.25 mg |
| CAPRYOL 90 | 295.50 mg |
| Mono-/Diglycerides of caprylic acid (CAPMUL MCM C8) | 147.75 mg |

### EXAMPLE 8

| | |
|---|---|
| Aliskiren | 15.00 mg |
| Polysorbat 80 (TWEEN 80) | 394.00 mg |
| PEG 300 | 98.50 mg |
| Propylene glycol di-caprylate/caprate (CAPTEX 200) | 328.33 mg |
| Mono-/Diglycerides of caprylic acid (CAPMUL MCM C8) | 164.17 mg |

### EXAMPLE 9

| | |
|---|---|
| Aliskiren | 15.00 mg |
| Polysorbat 80 (TWEEN 80) | 344.75 mg |
| PEG 300 | 98.50 mg |
| Propylene glycol di-caprylate/caprate (CAPTEX 200) | 361.17 mg |
| Mono-/Diglycerides of caprylic acid (CAPMUL MCM C8) | 180.58 mg |

### EXAMPLE 10

| | |
|---|---|
| Aliskiren | 15.00 mg |
| Vitamin E-TPGS | 394.00 mg |
| PEG 300 | 98.50 mg |
| Propylene glycol di-caprylate/caprate (CAPTEX 200) | 328.33 mg |
| Mono-/Diglycerides of caprylic acid (CAPMUL MCM C8) | 164.17 mg |

### EXAMPLE 11

| | |
|---|---|
| Aliskiren | 15.00 mg |
| Vitamin E-TPGS | 197.00 mg |
| PEG 300 | 98.50 mg |
| Propylene glycol di-caprylate/caprate (CAPTEX 200) | 459.67 mg |
| Mono-/Diglycerides of caprylic acid (CAPMUL MCM C8) | 229.83 mg |

### EXAMPLE 12

| | |
|---|---|
| Aliskiren | 15.00 mg |
| Vitamin E-TPGS | 295.50 mg |
| PEG 300 | 98.50 mg |
| Propylene glycol di-caprylate/caprate (CAPTEX 200) | 394.00 mg |
| Mono-/Diglycerides of caprylic acid (CAPMUL MCM C8) | 197.00 mg |

### EXAMPLE 13

| | |
|---|---|
| Aliskiren | 15.00 mg |
| Vitamin E-TPGS | 344.75 mg |
| PEG 300 | 98.50 mg |
| Propylene glycol di-caprylate/caprate (CAPTEX 200) | 361.17 mg |
| Mono-/Diglycerides of caprylic acid (CAPMUL MCM C8) | 180.58 mg |

What is claimed is:

1. A pharmaceutical composition for oral administration comprising a δ-amino-γ-hydroxy-ω-aryl-alkanoic acid amide renin inhibitor in an absorption enhancing carrier medium comprising:
(a) a lipophilic component;
(b) a high HLB surfactant; and
(c) a hydrophilic component;
which composition upon admixing forms a stable microemulsion preconcentrate.

2. A pharmaceutical composition according to claim 1, wherein the lipophilic component comprises a low HLB surfactant.

3. A pharmaceutical composition according to claim 2, wherein the lipophilic component comprises a low HLB surfactant which is a based on a medium or a long chain fatty acid, or a mixture of fatty acids thereof, and an oil which is a medium or a long chain fatty acid triglyceride, or a mixture of triglycerides thereof.

4. A pharmaceutical composition according to claim 3, wherein the lipophilic component comprises a low HLB surfactant which is based on a medium chain fatty acid, or a mixture of fatty acids thereof, and an oil which is a medium chain fatty acid triglyceride, or a mixture of triglycerides thereof.

5. A pharmaceutical composition according to claim 4, wherein the microemulsion preconcentrate is in the form of a water-in-oil microemulsion which upon administration or dilution with an aqueous medium spontaneously converts to an oil-in-water microemulsion.

6. A pharmaceutical composition according to claim 4, wherein the δ-amino-γ-hydroxy-ω-aryl-alkanoic acid amide renin inhibitor has the formula 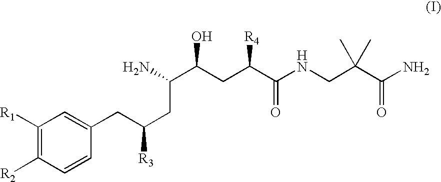
wherein R₁ is C₁₋₄alkoxy-C₁₋₄alkoxy or C₁₋₄alkoxy-C₁₋₄alkyl; R₂ is C₁₋₄alkyl or C₁₋₄alkoxy; and R₃ and R₄ are independently branched C₃₋₄alkyl; or a pharmaceutically acceptable salt thereof.

7. A pharmaceutical composition according to claim 6, wherein the δ-amino-γ-hydroxy-ω-aryl-alkanoic acid amide renin inhibitor is a compound of formula (I) wherein R₁ is 3-methoxypropoxy; R₂ is methoxy; and R₃ and R₄ are isopropyl; or a pharmaceutically acceptable salt thereof.

8. A pharmaceutical composition according to claim 7, wherein the δ-amino-γ-hydroxy-ω-aryl-alkanoic acid amide renin inhibitor is (2S,4S,5S,7S)-5-amino-4-hydroxy-2-isopropyl-7-[4-methoxy-3-(3-methoxy-propoxy)-benzyl]-8-methyl-nonanoic acid (2-carbamoyl-2-methyl-propyl)-amide hemifumarate.

9. A pharmaceutical composition according to claim 8, wherein the microemulsion preconcentrate is in the form of a water-in-oil microemulsion which upon administration or dilution with an aqueous medium spontaneously converts to an oil-in-water microemulsion.

10. A pharmaceutical composition according to claim 6, wherein the medium chain fatty acids of the lipophilic component have from 8 to 12 carbon atoms.

11. A pharmaceutical composition according to claim 10, wherein the oil is selected from propylene glycol di-caprylate/caprate and glyceryl tri-caprylate/caprate.

12. A pharmaceutical composition according to claim 6, wherein the low HLB surfactant has an HLB value ranging from about 2.5 to about 6.

13. A pharmaceutical composition according to claim 6, wherein the high HLB surfactant has an HLB value ranging from about 13 to about 20.

14. A pharmaceutical composition according to claim 13, wherein the high HLB surfactant is selected from polysorbat 80, macrogol-15 hydroxystearate, vitamin E-TPGS and polyoxyl 40 hydrogenated castor oil.

15. A pharmaceutical composition according to claim 6, wherein the hydrophilic phase comprises PEG 300.

16. A pharmaceutical composition according to claim 15, wherein the medium chain fatty acids of the lipophilic component have from 8 to 12 carbon atoms.

17. A pharmaceutical composition according to claim 16, wherein the low HLB surfactant has an HLB value ranging from about 2.5 to about 6.

18. A pharmaceutical composition according to claim 17, wherein the high HLB surfactant has an HLB value ranging from about 13 to about 20.

19. A pharmaceutical composition according to claim 18, wherein the δ-amino-γ-hydroxy-ω-aryl-alkanoic acid amide renin inhibitor is a compound of formula (I) wherein R₁ is 3-methoxypropoxy; R₂ is methoxy; and R₃ and R₄ are isopropyl; or a pharmaceutically acceptable salt thereof.

20. A pharmaceutical composition according to claim 19, wherein the oil is selected from propylene glycol di-caprylate/caprate and glyceryl tri-caprylate/caprate.

21. A pharmaceutical composition according to claim 19, wherein the high HLB surfactant is selected from polysorbat 80, macrogol-15 hydroxystearate, vitamin E-TPGS and polyoxyl 40 hydrogenated castor oil.

22. A pharmaceutical composition according to claim 19, wherein the δ-amino-γ-hydroxy-ω-aryl-alkanoic acid amide renin inhibitor is (2S,4S,5S,7S)-5-amino-4-hydroxy-2-isopropyl-7-[4-methoxy-3-(3-methoxy-propoxy)-benzyl]-8-methyl-nonanoic acid (2-carbamoyl-2-methyl-propyl)-amide hemifumarate.

23. A pharmaceutical composition according to claim 22, wherein the oil is selected from propylene glycol di-caprylate/caprate and glyceryl tri-caprylate/caprate.

24. A pharmaceutical composition according to claim 23, wherein the high HLB surfactant is selected from polysorbat 80, macrogol-15 hydroxystearate, vitamin E-TPGS and polyoxyl 40 hydrogenated castor oil.

25. A pharmaceutical composition according to claim 24, wherein the microemulsion preconcentrate is in the form of a water-in-oil microemulsion which upon administration or dilution with an aqueous medium spontaneously converts to an oil-in-water microemulsion.

26. A method for the treatment of hypertension, congestive heart failure, cardiac hypertrophy, cardiac fibrosis, cardiomyopathy postinfarction, complications resulting from diabetes, such as nephropathy, vasculopathy and neuropathy, diseases of the coronary vessels, restenosis following angioplasty, raised intra-ocular pressure, glaucoma, abnormal vascular growth, hyperaldosteronism, anxiety states and cognitive disorders which method comprises administering a therapeutically effective amount of a pharmaceutical composition according to claim 1 to a patient in need thereof.
